(19) United States
(12) Patent Application Publication
(10) Pub. No.: US 2010/0268028 A1
(43) Pub. Date: Oct. 21, 2010

(54) DEVICES AND METHODS FOR GUIDING SURGICAL INSTRUMENTS

(75) Inventor: Ragae M. Ghabrial, Loveland, OH (US)

Correspondence Address: Ethicon Endo-Surgery/Nutter, McClennen & Fish LLP, Seaport West, 155 Seaport Blvd., Boston, MA, 02210-2604, US

(73) Assignee: ETHICON ENDO-SURGERY, INC., Cincinnati, OH (US)

(21) Appl. No.: 12/427,276

(22) Filed: Apr. 21, 2009

(51) Int. Cl. A61B 1/01

(52) U.S. Cl. 600/114

(57) ABSTRACT

Methods and devices are provided for guiding surgical instruments during minimally invasive surgical procedures. In one embodiment, a surgical instrument is provided that includes a flexible, cannulated elongate shaft having an inner wall of variable thickness. The inner wall of the shaft can have a first thickness in a proximal portion of the device and can have a second, larger thickness in a distal portion of the instrument. The instrument can also include at least one guide mechanism configured to guide at least one surgical device through at least a portion of the cannulated interior of the elongate shaft.

### FIELD OF THE INVENTION

The present invention relates to devices and methods for guiding surgical instruments during minimally invasive surgical procedures.

### BACKGROUND OF THE INVENTION

Minimally invasive surgical techniques such as endoscopies and laparoscopies are often preferred over traditional open surgeries because the recovery time, pain, and surgery-related complications are typically less with minimally invasive surgical techniques. Rather than cut open large portions of the body in order to access inner cavities, such as the peritoneal cavity, surgeons either rely on natural orifices of the body or create one or more small orifices in which surgical instruments can be inserted to allow surgeons to visualize and operate at the surgical site. Surgeons can then perform a variety of diagnostic procedures, such as visual inspection or removal of a tissue sample for biopsy, or treatment procedures, such as removal of a polyp or tumor or restructuring tissue.

Because of the rise in popularity of minimally invasive surgeries, there has been significant development with respect to the instruments used in such procedures. These instruments need to be suitable for precise placement of a working end at a desired surgical site to allow the surgeon to see the site and/or perform the necessary actions at such site. Oftentimes the instruments either themselves contain a device that allows the surgeon to see the site or else the instruments are used in conjunction with an instrument that can provide visual assistance. At least one of these types of devices, an endoscope, is typically configured with both a lens to visualize the surgical site and a channel through which instruments can be delivered to the surgical site for subsequent use. The instruments themselves can be used to engage and or treat tissue and other portions within the body in a number of different ways to achieve a diagnostic or therapeutic effect.

Minimally invasive procedures typically require that the shaft of any device inserted into the body be flexible to navigate various, often small and nonlinear, shapes within the anatomy while still allowing stability and precision at the working end. During an endoscopy, for example, it can be necessary to navigate a device in a variety of different directions before the device reaches its desired destination, which means it is desirable that any such device be flexible. However, once the device reaches its desired destination, it can also be desirable that the device be strong and stable so that the surgeon can operate with precision. It can be difficult for a device to be sufficiently flexible to navigate through the body but be sufficiently strong to maintain structural integrity and steerability once navigated to its desired surgical location. Furthermore, natural orifices and surgical incisions used to introduce devices into a patient in a minimally invasive procedure are relatively small. Devices used in minimally invasive procedures thus typically need to have a relatively small size to be safely introduced into a patient. The small size of the devices can make them more difficult to navigate through the body with precision and ease.

It can also be desirable to have multiple devices concurrently inserted into a patient during a surgical procedure so the devices can cooperate with each other and/or be quickly used in succession, but the relatively small orifices typically used to introduce the devices can limit the size and number of devices concurrently introduced into a body. Multiple devices can be successively introduced into a patient during a surgical procedure to perform different aspects of a surgical procedure, but the repeated insertion and withdrawal of various instruments into a patient can increase chances of patient injury and increase the length of the surgical procedure.

Accordingly, there remains a need for improved devices and methods for controlling surgical devices used during surgical procedures.

### SUMMARY OF THE INVENTION

The present invention generally provides methods and devices for guiding surgical instruments during minimally invasive surgical procedures. In one embodiment, a surgical device is provided that includes a flexible elongate shaft having an inner lumen extending therethrough and a support structure coupled to an interior surface of the elongate shaft. A proximal portion of the elongate shaft has a wall with a first thickness, and a distal portion of the elongate shaft has a wall with a second thickness that is larger than the first thickness. The support structure longitudinally extends through the inner lumen of the elongate shaft along a longitudinal length of the elongate shaft.

The support structure can vary in any number of ways. In some embodiments, the support structure can include a flexible central column extending through the inner lumen in the proximal and distal portions of the elongate shaft. At least one strut can radially extend between the central column and the wall in the distal portion of the elongate shaft. A plurality of struts, each of the struts radially extending between the central column and the wall in the distal portion of the elongate shaft, can be spaced substantially equidistantly around the central column. The central column can have at least one cut-out formed therein that extends along a longitudinal length of the central column and that is configured to slidably guide a surgical tool therethrough. The surgical device can optionally include a mating member that is in sliding engagement with the central column. The surgical device can also include an accessory secured to the mating member and configured to receive a surgical instrument therein. The accessory and the mating member can be configured to slide as a unit along the central column. In some embodiments, the support structure can include at least one track extending through the inner lumen in the proximal and distal portions of the elongate shaft. The at least one track can be coupled to an interior surface of the walls in the distal and proximal portions and be configured to engage and guide a surgical instrument disposed through the inner lumen of the elongate shaft. The surgical device can also include an accessory that is configured to slide along the track within the inner lumen of the elongate shaft and that has a passageway for passing a surgical instrument.

The surgical device can vary in any other number of ways. For example, the first cross-sectional thickness can have a thickness that is in a range of about 10% to 50% of the second cross-sectional thickness. For another example, the proximal portion of the elongate shaft can be radially compressible.

In another embodiment, a surgical device is provided that includes an overtube having an insertion section with proximal and distal portions. The proximal portion has a flexible wall with a first thickness and a first compressive strength, and the distal portion has a flexible wall with a second thickness greater than the first thickness and a second compressive strength greater than the first compressive strength. A central column extends through the insertion section, is flexible, and is configured to transmit a distally directed force to maneuver the overtube axially. The surgical device can have any number of variations. For example, the overtube can have at least one strut extending radially between the central column and the flexible wall of the distal portion. In some embodiments, the overtube can have a plurality of radial struts extending between the central column and the flexible wall of the distal portion, the plurality of struts defining a plurality of channels in the distal portion equal to the number of struts. The central column can optionally have at least one cut-out formed in an outer surface thereof, the at least one cut-out configured to slidably guide a surgical tool therethrough. For another example, the surgical device can include at least one mating member that is coupled to the central column and that extends through the insertion section of the overtube. An accessory can be coupled to the mating member and extend radially relative to the central column. For yet another example, the central column can have a longitudinal track coupled to an exterior surface thereof, the track being configured to slidably guide a surgical tool therethrough. The central column can have a passageway extending longitudinally therethrough that is configured to slidably receive a surgical tool therein.

In another embodiment, a surgical device is provided that includes an overtube having an insertion section with a proximal portion and a distal portion. The proximal portion has a flexible wall with a first thickness and a first compressive strength, and the distal portion has a flexible wall with a second thickness greater than the first thickness and a second compressive strength greater than the first compressive strength. The surgical device also includes at least one track that is coupled to an interior surface of the overtube and that extends longitudinally along the insertion section. The at least one track is configured to slidably engage a surgical tool disposed through the overtube. The surgical device can vary in any number of ways. In some embodiments, the overtube can have a plurality of tracks coupled to an interior surface thereof. The tracks can be arranged substantially equidistantly around a circumference of the overtube.

In another aspect, a surgical method is provided that includes advancing an overtube into a patient. The overtube has a passageway extending therethrough, has a proximal portion with a wall having a first thickness, and has a distal portion having a wall with a second thickness that is larger than the first thickness. The overtube also has a support structure longitudinally extending through the passageway in the proximal and distal portions of the overtube. The surgical method also includes guiding a flexible surgical instrument through the overtube using the support structure. The method can vary in any number of ways. For example, guiding a flexible surgical instrument through the overtube using the support structure can include engaging the surgical instrument with a continuous track extending through the passageway of the overtube and advancing the surgical instrument along the continuous track. In some embodiments, the continuous track can be in the form of a cut-out formed in a central column longitudinally extending through the overtube and coupled to an interior surface of the wall in the distal portion of the overtube. In other embodiments, the continuous track can be in the form of a rail longitudinally extending through the overtube and coupled to an interior surface of the walls in the proximal and distal portions of the overtube.

### BRIEF DESCRIPTION OF THE DRAWINGS

The invention will be more fully understood from the following detailed description taken in conjunction with the accompanying drawings, in which:

FIG. 1A is a perspective view of one embodiment of a surgical device having a central column and an inner wall in a distal portion thereof that is thicker than an inner wall in a proximal portion thereof;

FIG. 1B is a perspective view of the device of FIG. 1A with the proximal portion radially collapsed inwards;

FIG. 2 is a partial cross-sectional view of the proximal and distal inner walls of the device of FIG. 1A;

FIG. 3 is a cross-sectional view of the distal portion of the device of FIG. 1A with surgical instruments received in an inner lumen of the device;

FIG. 4 is a cross-sectional view of the proximal portion of the device of FIG. 1A with surgical instruments received in the inner lumen of the device;

FIG. 5 is a perspective view of another embodiment of a surgical device having a central column with a plurality of cut-outs formed therein, and the device having an inner wall in a distal portion thereof that is thicker than an inner wall in a proximal portion thereof;

FIG. 6 is a cross-sectional view of the distal portion of the device of FIG. 5;

FIG. 7 is a cross-sectional view of a distal portion of another embodiment of a surgical device having a central column with a plurality of cut-outs formed therein with a guide member slidably seated in one of the cut-outs;

FIG. 8 is a side view of another embodiment of a surgical device having a plurality of tracks extending longitudinally therethrough and having an inner wall in a distal portion thereof that is thicker than an inner wall in a proximal portion thereof;

FIG. 9 is a side transparent view of the device of FIG. 8;

FIG. 10 is a cross-sectional view of the proximal portion of the device of FIG. 8;

FIG. 11 is a cross-sectional view of the proximal portion of the device of FIG. 8 with a guide member slidably received in one of the tracks;

FIG. 12 is a partial cross-sectional view of the proximal portion of the device of FIG. 8 with a guide member slidably received in one of the tracks;

FIG. 13 is a partial cross-sectional view of one embodiment of a rail mated to a mating member;

FIG. 14 is a partial cross-sectional view of another embodiment of a rail mated to a mating member;

FIG. 15 is a partial cross-sectional view of yet another embodiment of a rail mated to a mating member;

FIG. 16 is a partial cross-sectional view of one embodiment of a rail mated to a mating member having a channel formed therein;

FIG. 17 is a partial cross-sectional view of another embodiment of a rail mated to a mating member;

FIG. 18 is a partial perspective view of another embodiment of a surgical device having a plurality of tracks extending longitudinally therethrough with two surgical instruments slidably received in two of the tracks, and the device having an inner wall in a distal portion thereof that is thicker than an inner wall in a proximal portion thereof;

FIG. 19 is a cross-sectional view of a distal portion of one embodiment of a surgical device having a plurality of tracks extending longitudinally along an outer surface thereof, the device inserted in one embodiment of a flexible outer sheath;

FIG. 20 is a partial side view of a proximal portion of the outer sheath of FIG. 19 including stability threads;

FIG. 21 is a perspective partially transparent view of one embodiment of a surgical device having an inner wall in a distal portion thereof that is thicker than an inner wall in a proximal portion thereof transorally introduced into a patient; and

FIG. 22 is a perspective partially transparent view of one embodiment of a surgical device having an inner wall in a distal portion thereof that is thicker than an inner wall in a proximal portion thereof introduced through a trocar into an abdomen of a patient.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Various exemplary methods and devices are provided for guiding surgical instruments during minimally invasive surgical procedures. In general, a surgical instrument is provided that includes a flexible, cannulated elongate shaft having an inner wall of variable thickness. The inner wall of the shaft can have a first thickness in a proximal portion of the device and can have a second, larger thickness in a distal portion of the instrument. The distal portion of the instrument can thus have increased structural integrity over the proximal portion of the instrument such that the distal portion of the instrument can substantially maintain its shape while the proximal portion of the instrument can collapse or otherwise deform inwards. In this way, the proximal portion of the instrument can occupy less space in a body of a patient and/or less space in an introducer device configured to introduce the instrument into a body of a patient through a natural or artificial access opening, thereby decreasing chances of the instrument harming the patient's body and/or allowing additional surgical devices to be inserted into a patient's body concurrent with the instrument. In an exemplary embodiment, the instrument can include at least one support structure configured to guide at least one surgical device through at least a portion of the cannulated interior of the elongate shaft to help smoothly guide the at least one surgical device through the instrument. The guide mechanism can also help position the at least one surgical device in a predictable position relative to the instrument and to any other surgical devices advanced through the instrument, thereby helping to reduce interference between multiple surgical devices positioned inside and/or outside the instrument.

A person skilled in the art will appreciate that while the methods and devices are described in connection with endoscopic procedures in which the surgical device is delivered through a natural orifice, the methods and devices disclosed herein can be used in numerous surgical procedures and with numerous surgical instruments. By way of non-limiting example, the devices can be used in laparoscopic procedures, in which the device is introduced percutaneously. The methods and devices can also be used in open surgical procedures. Furthermore, the surgical device can be configured to pass through any portion of a body, but in an exemplary embodiment, the surgical device is configured to pass through a tortuous pathway. A person skilled in the art will appreciate that the term “tortuous pathway” as used herein is intended to include a tubular body lumen or organ, e.g., the colon or esophagus. While the methods and devices disclosed herein are described in connection with steering a scoping device, e.g., an endoscope, a laparoscope, and a colonoscope, a person skilled in the art will also appreciate that the methods and devices disclosed herein can be used with any surgical instrument configured to be inserted into a body, such as through a natural orifice, through a puncture hole formed in tissue, and in any other way appreciated by a person skilled in the art. While the surgical instrument can be rigid and/or flexible, in an exemplary embodiment, at least a proximal portion of the surgical instrument is flexible.

The devices discussed herein can be made from any combination of rigid and/or flexible materials, but in an exemplary embodiment the materials are biocompatible and suitable for use in surgical procedures. A person skilled in the art will appreciate that the term “flexible” as used herein is intended to encompass a variety of configurations. Generally, a “flexible” member is one which, to at least some degree of elasticity is capable of bending or deforming without breaking. In an exemplary embodiment, the device or at least portions thereof are composed of at least one biocompatible and flexible material, e.g., plastic, titanium, stainless steel, etc.

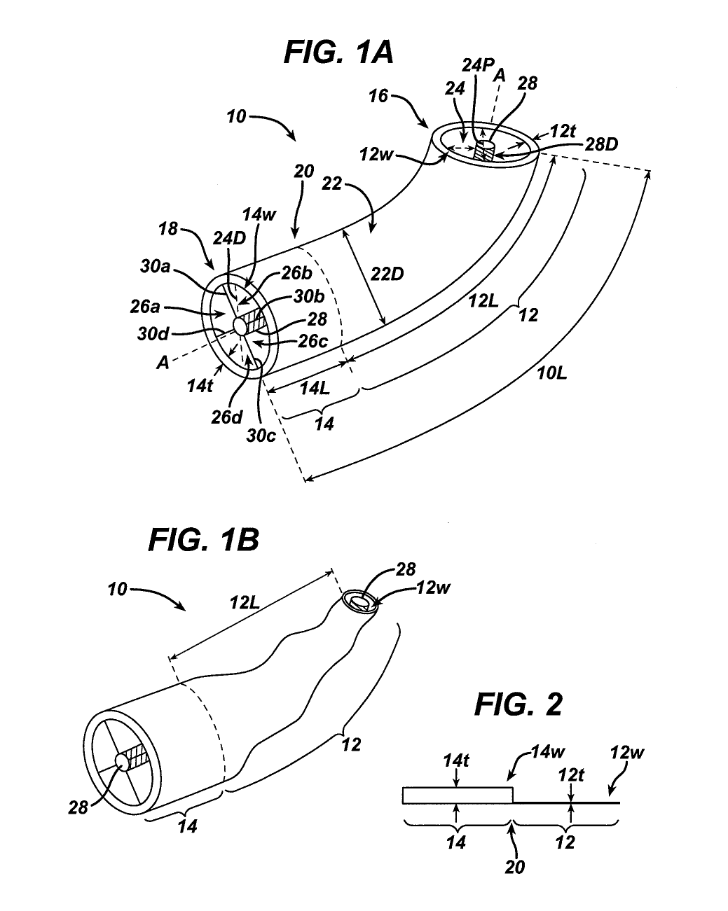

FIGS. 1A, 1B, and 2 illustrate one exemplary embodiment of a surgical device 10 effective for guiding one or more additional surgical tools therethrough and into a body of a patient. Generally, the device 10 has a flexible, cannulated elongate shaft or overtube 22 with proximal and distal portions 12, 14 having different sized proximal and distal inner walls 12w, 14w. The shaft 22 has an inner lumen 24 extending between proximal and distal ends 16, 18 thereof. In an exemplary embodiment, the device 10 can include a support structure configured to help guide one or more surgical devices through the inner lumen 24 of the shaft 22. Generally, the support structure can be coupled to an interior or inner surface of the shaft 22 and can longitudinally extend through the inner lumen 24 along a longitudinal length 10L of the device 10, which in this embodiment corresponds to the longitudinal length of the shaft 22. The support structure can have a variety of configurations. In this illustrated embodiment, the support structure includes a flexible central column 28 that can be axially aligned with a central longitudinal axis A of the device 10 and longitudinally extend through the inner lumen 24 along any portion of a longitudinal length 10L of the device 10, e.g., along an entire longitudinal length 10L of the device 10 as shown. The device 10 can also include one or more struts 30a, 30b, 30c, 30d radially extending between the central column 28 and the proximal inner wall 12w and/or the distal inner wall 14w that are configured to partition the inner lumen 24 into a plurality of channels 26a, 26b, 26c, 26d in at least in a portion of the device 10, e.g., in the distal portion 14 of the device 10 as shown.

The shaft 22 can have any size, shape, and configuration, as will be appreciated by a person skilled in the art. The shaft 22 can be rigid, flexible, or a combination thereof, but in this illustrated embodiment it is flexible along its longitudinal length that corresponds to the longitudinal length 10L of the device. The shaft 22 can vary in longitudinal length depending on the device's intended application. The proximal and distal portions 12, 14 of the shaft 22 can also each have any respective longitudinal length 12L, 14L along the shaft 22 that added together equal the device's longitudinal length 10L. In an exemplary embodiment, the device 10 can have a longitudinal length 10L of about 100 cm, the proximal portion 12 can have a longitudinal length 12L of about 90 cm between the proximal end 16 of the device 10 and an intermediate point 20 of the device 10 located between the proximal and distal ends 16, 18, and the distal portion 14 can have a longitudinal length 14L of about 10 cm between the distal end 18 of the device 10 and the intermediate point 20 of the device 10.

The shaft 22 can be formed from a single component or multiple segments, and can be coiled or non-coiled. The flexibility of the shaft 22, as well as a relatively small diameter of its inner lumen 24, can allow the shaft 22 to be used in endoscopic procedures, whereby the device 10 is introduced translumenally through a natural or artificial orifice. In an exemplary embodiment, the shaft 22 can be substantially cylindrical, which can help ease the device's passage into and through the body and prevent the shaft 22 from harming or getting caught on tissue.

The shaft 22 can have a uniform or non-uniform outer diameter 22D along its longitudinal length. In this illustrated embodiment, the shaft 22 has a uniform outer diameter 22D along its longitudinal length but has a variable inner diameter, e.g., the inner lumen 24 has a variable diameter between the proximal and distal ends 16, 18 of the shaft 22.

As mentioned above, the inner wall of the shaft 22 can have a variable thickness in the shaft's proximal and distal portions 12, 14 with the distal inner wall 14w having a larger thickness 14t than a thickness 12t of the inner wall 12w in the proximal portion 12. The different sized inner walls 12w, 14w can thus cause the inner lumen 24 to have a first diameter 24P in the proximal portion 12 and a second, smaller diameter 24D in the distal portion 14. The proximal and distal walls 12w, 14w can each have any thickness, but in an exemplary embodiment, the proximal inner wall 12w can have a thickness 12t that is about 25% of the distal inner wall thickness 14t, e.g., the shaft 22 having a proximal inner wall thickness 12t of about 0.25 mm and a distal inner wall thickness 14t of about 1 mm. As illustrated in this embodiment the thicknesses 12t, 14t of the inner walls 12w, 14w can be substantially constant along their respective longitudinal lengths 12L, 14L, although in some embodiments one or both of the thicknesses 12t, 14t of the inner walls 12w, 14w can vary along their respective longitudinal lengths 12L, 14L. Substantially at the intermediate point 20 where the proximal and distal portions 12, 14 meet, the inner wall of the shaft 22 can gradually taper or slope from the larger inner wall 14w thickness 14t to the smaller inner wall 12w thickness 12t around an inner circumference of the inner lumen 24. Alternatively, as illustrated in this embodiment in FIG. 2, the disparate thicknesses 12t, 14t of the proximal and distal inner walls 12w, 14w, respectively, can abruptly meet without a taper or slope at the intermediate point 20 around the inner circumference of the inner lumen 24.

With the shaft 22 being flexible in this illustrated embodiment and having the distal inner wall 14w thicker than the proximal inner wall 12w, the distal portion 14 of the shaft 22 can thereby have a compressive strength that is greater than a compressive strength of the proximal portion 12. In this way, the shaft 22 can have enough structural integrity to be advanced through a body, alone or through an introducer device, e.g., a cannula, and to receive at least one surgical instrument in its inner lumen 24. The proximal portion 12 of the shaft 22 can nevertheless be configured to be radially compressible, as illustrated in one embodiment in FIG. 1B, such that the proximal inner wall 12w can collapse or move inwards to reduce the inner diameter 24P of the inner lumen 24 in the proximal portion 12, thereby allowing the proximal portion 12 of the shaft 22 to more easily fit inside and to exert less pressure on a patient's body, e.g., within a body lumen such as the esophagus, when the distal portion 14 is positioned at a surgical site, e.g., in a stomach of a patient. The proximal inner wall 12w can uniformly radially collapse along the longitudinal length 12L of the proximal portion 12, but in an exemplary embodiment, the proximal inner wall 12w can nonuniformly radially collapse along its longitudinal length 12L. The proximal portion 12 can thus adapt to the body lumen or other structure in which it is inserted, radially collapsing to any degree along its longitudinal length 12L as allowed by the diameter of the body lumen or other structure in which it is inserted. In some embodiments, the proximal inner wall 12w can radially collapse inwards such that the proximal inner wall 12w contacts the central column 28. One or more surgical devices can still be slidably movable through the inner lumen 24 in the proximal portion 12 when the proximal portion 12 is at least partially radially collapsed, with the proximal inner wall 12w being configured to flex or move radially outward as necessary to accommodate one or more surgical devices being moved therethrough. Being thicker and having a greater compressive strength, the distal portion 14 can help the device 10 be advanced through a patient's body and help substantially maintain the inner diameter 24D of the lumen 24 when the distal portion 14 is disposed in a body cavity, e.g., the abdominal cavity, such that the distal portion 14 can be configured to slidably receive one or more surgical devices in the inner lumen 24 in the distal portion 14 and allow the one or more surgical devices received therein to advance distally beyond the device's distal end 18 to access the body cavity.

As mentioned above, the shaft's inner lumen 24 can be configured to receive at least one surgical instrument therein. The inner lumen 24 can be a cannulated tunnel having any size and shape, but in an exemplary embodiment, the inner lumen 24 is substantially cylindrical and is sized to receive at least one surgical instrument such that the surgical instrument can be slidably movable therein. As mentioned above, at least a portion of the inner lumen 24 can be partitioned to have the channels 26a, 26b, 26c, 26d, which can each be configured to receive and guide at least one surgical instrument through the device 10 in a predictable position relative to the shaft 22 and to any other surgical devices received in the inner lumen 24. Generally, the channels 26a, 26b, 26c, 26d can be defined by the central column 28 and the struts 30a, 30b, 30c, 30d. The central column 28, the struts 30a, 30b, 30c, 30d, and the channels 26a, 26b, 26c, 26d can have a variety of sizes, shapes, and configurations, and are further discussed below.

The central column 28 can be rigid, flexible, or a combination thereof, but as shown in this illustrated embodiment it can be, like the shaft 22, flexible along its longitudinal length that corresponds to the longitudinal length 10L of the device 10, although the column 28 can have any longitudinal length greater or less than the shaft 22 depending on the device's intended application. In an exemplary embodiment, the column 28 can be formed of a flexible material such as polyurethane or polycarbonate to allow the column 28 to flex while also resisting longitudinal compression or buckling. The column 28 can thus be configured to transmit a distally directed force to maneuver the shaft 22 axially, e.g., to advance the shaft 22 through and/or withdraw the shaft 22 from a body. The column 28 can have any cross-sectional shape, e.g., circular (as shown), ovular, rectangular, triangular, etc., that can be the same or variable along the column's longitudinal length. In an exemplary embodiment the column 28 has a shape corresponding to the shape of the shaft's inner lumen 24, e.g., both cylindrical as shown. The column 28 can also have any diameter 28D, which can be the same or different along the column's longitudinal length. In an exemplary embodiment, the column 28 can have a substantially constant diameter 28D, e.g., about 2 mm. The column 28 can be formed from a single component or multiple segments, and can be coiled or non-coiled. Although the column 28 is shown as a solid member, the column 28 can have a central passageway extending longitudinally therethrough or have any number of hollow portions. A central passageway extending longitudinally through a central column can be configured to slidably receive one or more surgical instruments therein.

The struts 30a, 30b, 30c, 30d can also be rigid, flexible, or a combination thereof, but in an exemplary embodiment the struts 30a, 30b, 30c, 30d can be substantially rigid to help provide structural integrity portion of the shaft 22 in which they are located, e.g., the distal portion 14. The struts 30a, 30b, 30c, 30d can have any shape and size same or different from any one or more of the other struts 30a, 30b, 30c, 30d, but in an exemplary embodiment each of the struts 30a, 30b, 30c, 30d can include a linear rod extending substantially perpendicular to the longitudinal axis A of the device between the column 28 and the distal inner wall 14w adjacent to the distal end 18 of the device 10. As will be appreciated by a person skilled in the art, although four struts 30a, 30b, 30c, 30d are shown defining four channels 26a, 26b, 26c, 26d of substantially equal size, the number and location of the struts 30a, 30b, 30c, 30d can vary. In an exemplary embodiment the struts 30a, 30b, 30c, 30d are spaced substantially equidistantly radially around a circumference of the column 28, as shown, to maximize a size of each of the channels 26a, 26b, 26c, 26d. The struts 30a, 30b, 30c, 30d can thus be configured as barriers between the channels 26a, 26b, 26c, 26d to help prevent interference between surgical devices received in different ones of the channels 26a, 26b, 26c, 26d and to more accurately position surgical devices advanced through the inner lumen 24 and distally beyond the device's distal end 18. In an exemplary embodiment, the struts 30a, 30b, 30c, 30d can be integrally formed with the column 28 and the distal inner wall 14w to help prevent the struts 30a, 30b, 30c, 30d from loosening or detaching during use of the device 10.

A person skilled in the art will also appreciate that while the struts 30a, 30b, 30c, 30d in this illustrated embodiment are located in the distal portion 14 of the device 10 to define the channels 26a, 26b, 26c, 26d in the distal portion 14, the struts 30a, 30b, 30c, 30d can be located in the distal and/or proximal portions 14, 12 of the device 10. In some embodiments, the struts 30a, 30b, 30c, 30d can be configured as planar members extending between the column 28 and the distal inner wall 14w along substantially the entire longitudinal length 14L of the distal portion 14 to help more distinctly define the channels 26a, 26b, 26c, 26d in the distal portion 14 and/or along at least a portion of each of the proximal and distal longitudinal lengths 12L, 14L to help define the channels 26a, 26b, 26c, 26d in both the proximal and distal portions 12, 14.

In this illustrated embodiment a number of the channels 26a, 26b, 26c, 26d equals a number of the struts 30a, 30b, 30c, 30d such that each of the channels 26a, 26b, 26c, 26d is defined by two of the struts 30a, 30b, 30c, 30d, but in other embodiments, the number of channels and struts can differ, e.g., the channels 26a, 26b, 26c, 26d can be defined by more than two struts 30a, 30b, 30c, 30d such as if two or more radially aligned struts extend between the column 28 and the inner wall of the shaft 22.

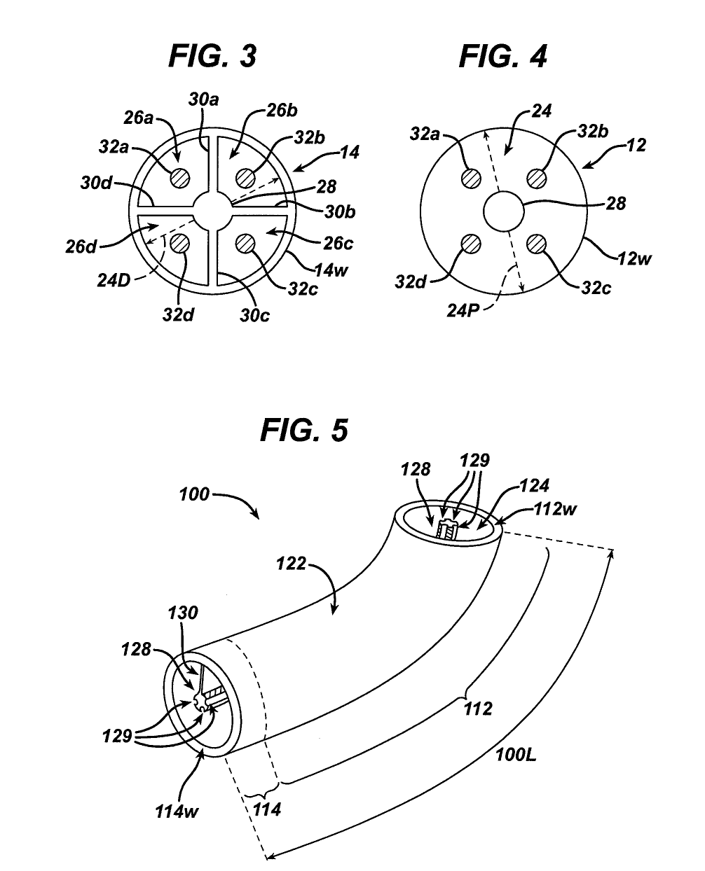

Any surgical device such as a grasper, a scoping device, a cutting instrument, etc., can be slidably received within the instrument's inner lumen 24. A person skilled in the art will appreciate that the term “grasper” as used herein is intended to encompass any surgical instrument that is configured to grab and/or attach to tissue and thereby manipulate the tissue, e.g., forceps, retractors, movable jaws, magnets, adhesives, stay sutures, etc. A person skilled in the art will also appreciate that the term “cutting instrument” as used herein is intended to encompass any surgical instrument that is configured to cut tissue, e.g., a scalpel, a harmonic scalpel, a blunt dissector, a cautery tool configured to cut tissue, scissors, an endoscopic linear cutter, a surgical stapler, etc. As shown in FIGS. 3 and 4, one or more surgical devices 32a, 32b, 32c, 32d can be received in the inner lumen 24 of the device 10 and extend through the proximal and distal portions 12, 14 of the device 10. Although four devices 32a, 32b, 32c, 32d are shown inserted in the device 10, one positioned in each of the channels 26a, 26b, 26c, 26d, a person skilled in the art will appreciate that any number of surgical devices can be simultaneously or sequentially received in the device 10 in any one or more of the channels 26a, 26b, 26c, 26d.

A person skilled in the art will appreciate that the device 10 can include a proximal handle (not shown) configured to facilitate grasping of the device 10. The handle can have a variety of sizes, shapes, and configurations and be attached to any part(s) of the device 10, e.g., in the proximal portion 12. Non-limiting examples of the handle include finger loops, knobs, an enlarged grip, etc. The handle can form a non-insertion section of the device 10, e.g., a section of the device 10 not configured to be inserted into a body of a patient, while a remainder of the device 10, e.g., the shaft 22, can form an insertion section of the device 10, e.g., a section of the device 10 configured to be inserted into a body of a patient.

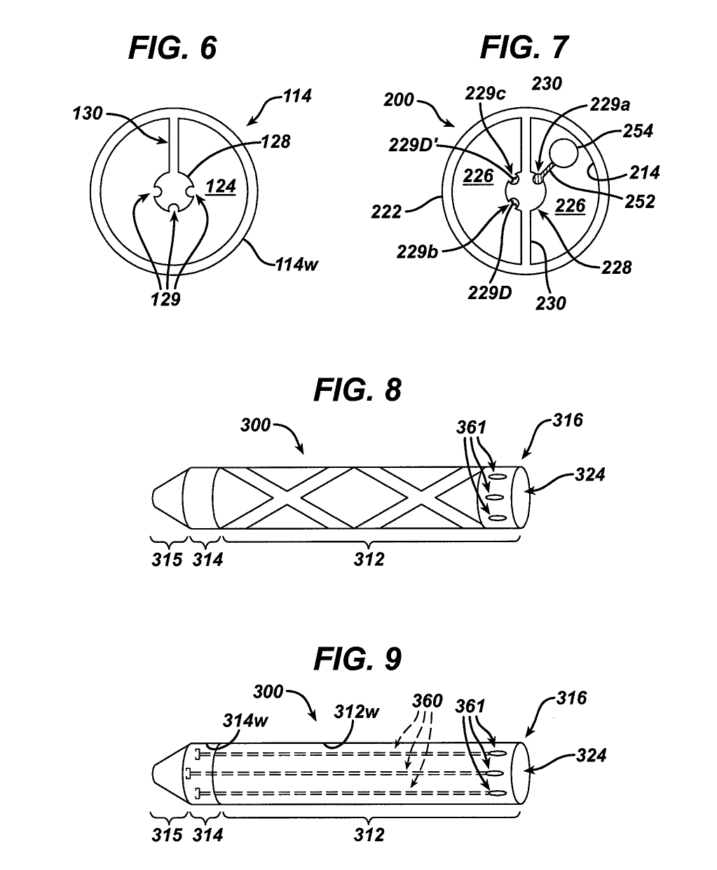

FIGS. 5 and 6 illustrate another exemplary embodiment of a surgical device 100 effective for guiding one or more additional surgical tools therethrough and into a body of a patient. Generally, the device 100 can be similar to the device 10 discussed above and include an elongate shaft or overtube 122 having an inner lumen 124 extending therethrough and having proximal and distal portions 112, 114 where an inner wall 112w of the proximal portion 112 can be thinner than an inner wall 114w of the distal portion 114. The device 100 can, also similar to the device 10, include a central column 128 attached to the shaft 122 with at least one strut 130 extending between the column 128 and an inner wall of the shaft 122, e.g., the distal inner wall 114w of the shaft 122. However, in this illustrated embodiment, the column 128 has at least one cut-out 129 formed in an outer surface of the column 128.

Generally, the cut-outs 129 can each be configured to slidably guide a surgical instrument therethrough, as discussed further below. Although three cut-outs 129 are shown, a person skilled in the art will appreciate that the column 128 can have any number of cut-outs 129 formed therein. The cut-outs 129 can have any size, shape, and configuration. The cut-outs 129 can longitudinally extend along any portion of the column 128. As in the illustrated embodiment, the cut-outs 129 can continuously extend along the entire longitudinal length of the column 128, which in this embodiment equals a longitudinal length 100L of the device 100. The cut-outs 129 can have any cross-sectional shape, e.g., c-shaped forming a substantially circular cut-out, and have any size configured to slidably mate with a surgical instrument including a corresponding protrusion configured to be seated in one of the cut-outs 129. The cut-outs 129 in this illustrated embodiment all have the same cross-sectional shape and all extend the longitudinal length of the column 128, but any of the cut-outs 129 can be same or different from any one or more of the other cut-outs 129. The cut-outs 129 can be spaced radially around a circumference of the column 128, equidistantly or nonequidistantly, as shown in this illustrated embodiment.

As shown in another exemplary embodiment in FIG. 7, a surgical device 200 similar to the devices 10, 100 can include an elongate shaft or overtube 222 and a central column 228 having first, second, and third cut-outs 229a, 229b, 229c formed therein and extending along a longitudinal length of the column 228. As shown in this illustrated embodiment, the first and second cut-outs 229a, 229b can each have a first diameter 229D, while the third cut-out 229c can have a second diameter 229D′ that is smaller than the first diameter 229D. One or more struts, e.g., two struts 230, can extend between the column 228 and a distal inner wall 214 of the device 200 to attach the column 228 to the shaft 222 and to define one or more channels, e.g., two channels 226, through which surgical instruments can pass. Also similar to the device 100, the distal inner wall 214 can be thicker than a proximal inner wall (not shown) of the device 200.

A surgical instrument 240 can be slidably received in one of the plurality of cut-outs 229 in a variety of ways. In some embodiments, a surgical instrument can include a protrusion on an outer surface thereof that is configured to be slidably seated in one of the cut-outs 229. In the illustrated embodiment, a guide member including a mating member 252 and an accessory 254 can be slidably received in one of the cut-outs 229. Various non-limiting embodiments of a guide member including a mating member and an accessory can be found in U.S. Patent Publication No. 2004/0230095 titled “Medical Apparatus For Use With An Endoscope” filed May 16, 2003, which is hereby incorporated by reference in its entirety. The guide member can be flexible, rigid, or any combination thereof. In an exemplary embodiment, the mating member 252 and the accessory 254 can be rigid, and the flange 256 can be flexible. One suitable material from which the flange 256 can be formed is a thermoplastic elastomer, such as a material designated commercially as Telcar 1025-75 (available from Teknor-Apex, Pawtucket, R.I.).

Generally, the accessory 254 can be in the form of a flexible tubular guide configured to receive and guide a surgical instrument therethrough. The mating member 252 can be configured to facilitate introduction of accessory 254 through the device 200 by providing a means to slide the accessory 254 along the longitudinal length of the column 228 in a controlled manner. The guide member can have any size, shape, and configuration, although in an exemplary embodiment the mating member 252 can have a longitudinal length substantially equal to or greater than a longitudinal length of the central column 228 to allow the mating member 252 to slide along an entire length of the column 228 such that a surgical instrument can be guided along the entire length of the column 228, and the accessory 252 can extend radially relative to the column 228 when the guide member is attached thereto. The mating member 252 and the accessory 254 can be formed as a unitary piece, or they can joined together by any suitable attachment method, as will be appreciated by a person skilled in the art.

The mating member 252 can be operatively coupled to the first cut-out 229a, or any of the other cut-outs 229b, 229c, through interlocking shapes or contours. A shape of the mating member 252 can have a substantially matching shape to a shape of the first cut-out 229a, e.g., both substantially cylindrical as illustrated in this embodiment, so that the mating member 252 can slide along the column 228. Although not shown in this embodiment, there can be a nominal clearance distance between a mating surface of the mating member 252 and a corresponding mating surface of the first cut-out 229a, so that no binding or pinching occurs when sliding one relative to the other. One or both of the column 228 and the mating member 252 can be formed from a flexible, low friction (“slippery”), plastic material, such as polyethylene, Teflon, or polypropylene to provide a low coefficient of friction between the column 228 and the mating member 252 as they slide relative to one another.

In use, once the device 200 has been advanced into a body of a patient, the mating member 252 can be engaged with the first cut-out 229a, or any of the other cutouts 229b, 229c, and the mating member 252 and the accessory 254 can then be advanced any distance along the longitudinal length of the column 228 by sliding engagement with the first cut-out 229a. In an exemplary embodiment, the accessory 254 can be positioned in or near a field of view in a body cavity, e.g., distal to a distal end of the device 200, to perform treatment or diagnosis, such as by advancing a surgical instrument through the accessory 254. A surgical instrument can be engaged with the accessory 254 and guided through an inner lumen of the shaft 222 before and/or after the accessory 254 has been coupled to the device 200.

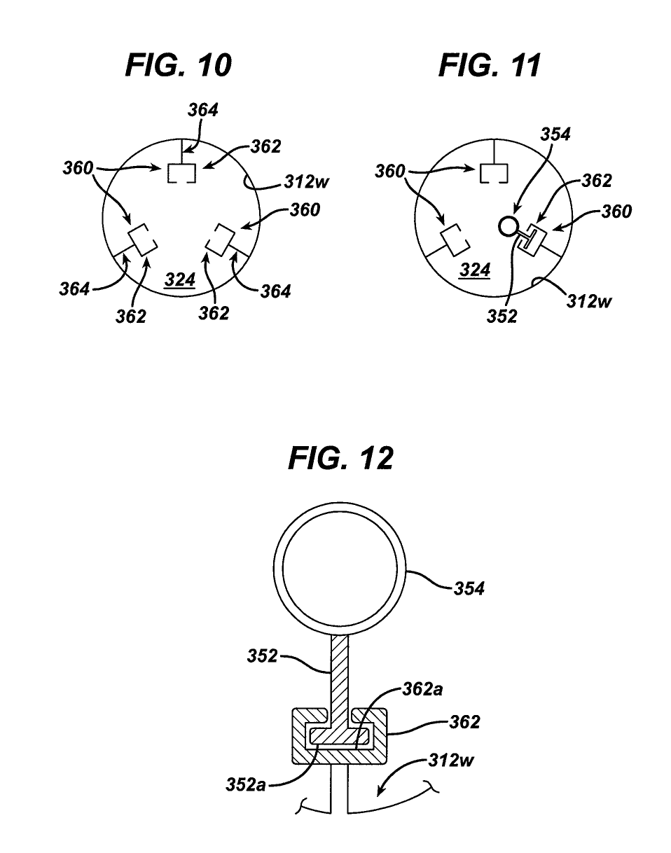

FIGS. 8-10 show another exemplary embodiment of a surgical device 300 effective for guiding one or more additional surgical tools therethrough and into a body of a patient. Generally, the device 300 can be similar to the devices 10, 100, 200 discussed above and include an elongate shaft or overtube 322 having an inner lumen 324 extending therethrough and having proximal and distal portions 312, 314 where an inner wall 312w of the proximal portion 312 can be thinner than an inner wall 314w of the distal portion 314. However, unlike the devices 10, 100, 200, the device 300 includes a tapered end cap 315 attached, removably or fixedly, to a distal end of the distal portion 314 of the device 300. The end cap 315 can generally be configured to facilitate insertion of the device 300 through an introducer device, a tortuous pathway, or other structure and to help distally direct surgical devices advanced through the device 300.

Also, unlike the devices 10, 100, 200, the device 300 in this illustrated embodiment does not include a support structure in the form of a central column extending therethrough. The device 300 instead includes a support structure in the form of at least one track 360 extending through the inner lumen 24 in the proximal and distal portions 312, 314 of the shaft 322. Although the device 300 is shown including three tracks 360 equidistantly spaced around a circumference of the device 300 in the inner lumen 324, the device 300 can include any number of tracks arranged in any way in the inner lumen 324. The tracks 360 can have any size, shape, and configuration, same or different from any one or more of the other tracks 360. The tracks 360 can longitudinally extend along any portion of the shaft 322 and/or the end cap 315. As in the illustrated embodiment, the tracks 360 can continuously extend along the substantially the entire longitudinal lengths of the proximal and distal portions 312, 314.

The device 300 can optionally include openings 361 formed in a sidewall thereof that are configured to allow passage of surgical instruments therethrough. The openings 361 can have any size, shape, and configuration. Although three oblong openings 361 are shown, the device 300 can have any number of openings 361, and the openings 361 can have any shape same or different from any of the other openings 361. Each of the openings 361 can be associated with one of the tracks 360 such that a surgical instrument inserted through one of the openings 361 can engage and slide through the track 360 associated with that opening 361. The openings 361 can thus facilitate engagement of surgical instruments with the tracks 360 by easing distal advancement of a surgical instrument into the lumen 324 and into engagement with a track 360. The openings 361 can be particularly helpful in embodiments such as the one illustrated in FIGS. 8-10 where the tracks 360 terminate distal to a proximal end 316 of the device 300 such that a proximal end of the tracks 360 can be difficult to locate.

As shown, the tracks 360 can include a rail 362 and a flange 364. One terminal end of the flange 364 can attach to an inner surface of the device 300, e.g., be molded to inner walls 312w, 314w of the proximal and distal portions 312, 314, while the other terminal end of the flange 364 can attach to the rail 362. The flanges 364 can be flexible to allow surgical devices to be seated in the rails 362 with increased freedom of movement. The rails 362 can be configured similar to the column cut-outs 129, 229 discussed above and be configured to slidably receive a surgical instrument therein and allow the surgical instrument to be glided therethrough. As shown in one embodiment in FIGS. 11 and 12, a guide member including a mating member 352 and an accessory 354, respectively similar to the mating member 252 and the accessory 354 discussed above, can be configured to be seated in the track 360 and slid along the track 360 with a contract surface 352a of the mating member 352 adjacent to and/or engaging with a contact surface 362a of the rail 362. As discussed above, the mating member 352 and the track 360 can have a variety of shapes, sizes, and configurations. As illustrated, the mating member 352 can include a distal member having a generally circular cross-section configured to be disposed in the track 360 having a generally rectangular cross-section. The distal member can have a diameter sized relative to the size of the track 360 such that the mating member 362 can slide in the track 360 while being maintained from radially disengaging from the track 360, e.g., by being removable from the track 360 only through longitudinal sliding the mating member 352 proximally through the track's associated opening 361. Also as discussed above, a surgical instrument can be advanced through a passageway in the accessory 354 and moved through the inner lumen 324.

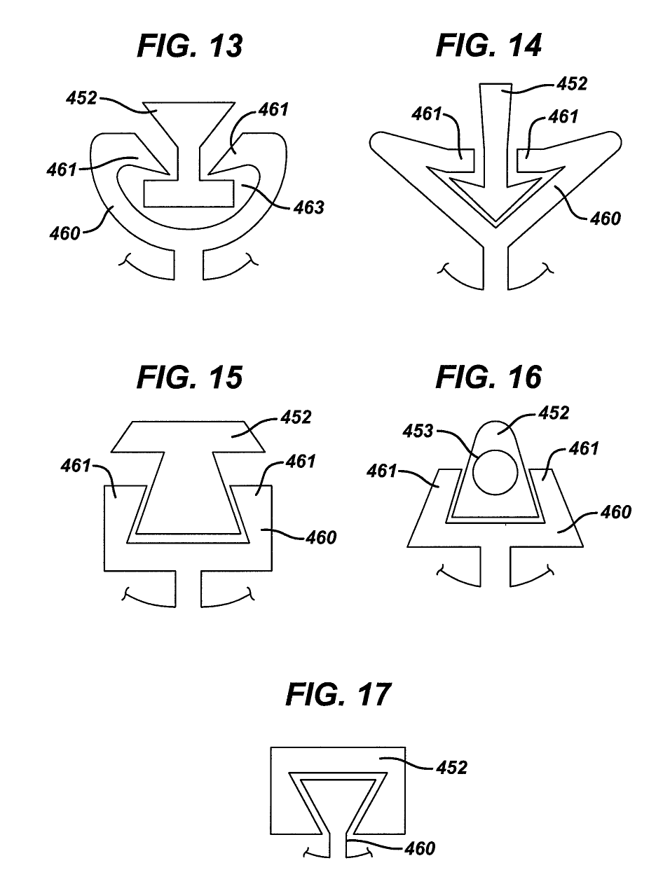

FIGS. 13-17 illustrate other exemplary embodiments of tracks 460 that can be attached to an inner surface of a surgical device and complementary guide members including mating members 452 configured to be slidably engaged with and guided along their complementary tracks 460. It will be understood that the tracks 460 and the mating members 452 can take on various shapes and configurations such that the mating members 452 can interlock with the shape of their complementary tracks 460 to allow sliding of the mating members 452 along their complementary tracks 460. The mating members 452 shown in FIGS. 13-17 can be formed on or otherwise attached to an outer surface of a surgical instrument to help slide the surgical instrument along a track 460, or any of the mating members 452 can be configured to couple to an accessory as discussed above. As viewed in cross section, the tracks 460 can have opposing arms 461 configured to maintain engagement of the mating member 452 with the track 460. The arms 461, together with a body of the track 460, can define a rail cavity 463 in which the mating member 452 can slide. If desired, the arms 461 can be provided with a desired level of resilience, such as by material choice or dimensioning, so that the mating member 452 can be caused to disengage from the track 460, e.g. by “unzipping” from the track 460, such as if the mating member 452 is urged radially outwardly from the rail cavity 463.

The embodiment shown in FIG. 16 incorporates an accessory 454 in the form of a circular working channel within the mating member 452. This embodiment can allow the passage of accessories within a lumen of the mating member 452 that slides inside the track 460. For example, the track 460 can have a substantially triangular recessed cross section that interlocks with the mating member 452 having an outer contour of a triangular shape, and a surgical instrument can slide within a lumen 453 located within the mating member 452. This particular embodiment can be suitable for a traumatic passage of the mating member 452 along the track 460, and when passing standard size accessories with the surgical device.

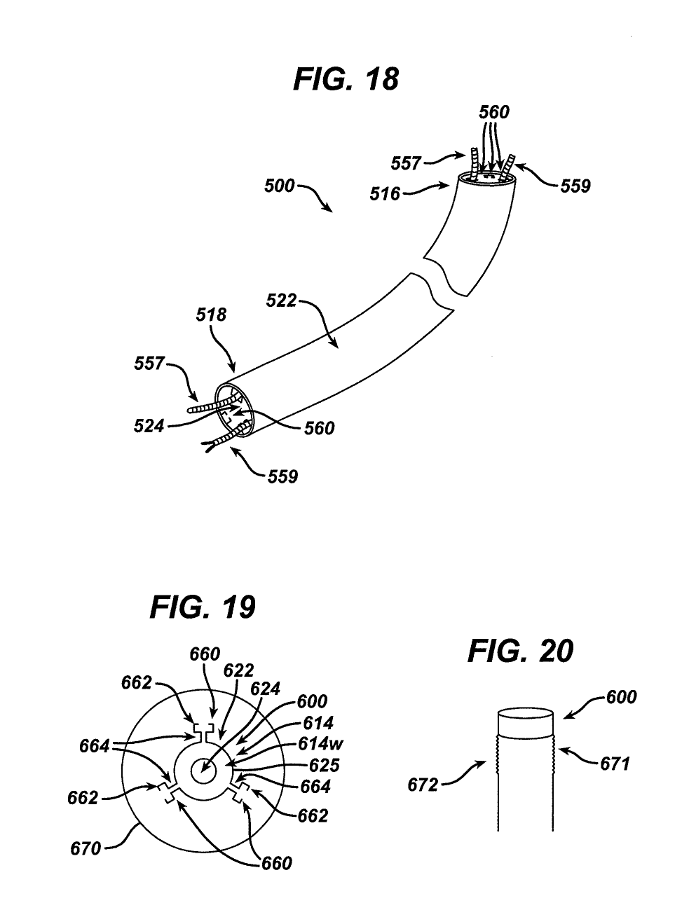

In some embodiments, at least one track configured to guide a surgical instrument through an inner lumen of a surgical device can have a low profile, e.g., have a rail in a direct contact with a surface of the inner lumen without a flange coupled therebetween. Such a low profile track can help conserve space within the inner lumen such that larger and/or more surgical instruments can be more easily inserted therethrough. FIG. 18 illustrates another embodiment of a surgical device 500, the device 500 including at least one low profile track 560 extending through an inner lumen 524 of an elongate shaft or overtube 522 of the device 500. The device 500 can otherwise be similar to the devices 10, 100, 200, 300 discussed above with a radially compressible proximal portion and be effective for guiding one or more additional surgical tools therethrough and into a body of a patient. Although the device 500 in this embodiment includes three tracks 560, the device 500 can include any number of tracks 560, with or without flanges coupled to rails. Also, as mentioned above, although the tracks 560 are shown with a retractor 557 guided through one of the tracks 560 and a pair of grasper jaws 559 inserted through another one of the tracks 560, with the retractor 557 and the jaws 559 each extending distally beyond the device's distal end 518 and proximally beyond the device's proximal end 516, any number and any type of surgical instrument can be advanced through any of the tracks 560.

Although the tracks discussed above are coupled to an inner surface of the elongate shaft within the inner lumen, in some embodiments one or more tracks can be coupled to an outer surface of the elongate shaft and radially extend outward therefrom. The tracks, having rails with or without flanges coupling the rails to an outer surface of the shaft, can otherwise be configured in any way as discussed above. Having one or more tracks coupled to an outer rather than inner surface of the shaft can help provide an unobstructed inner lumen for a surgical device, such as a scoping device that can be configured to provide visualization of the device's insertion into a body and/or of any surgical devices glided through any of the outer surface tracks. FIG. 19 illustrates one embodiment of a surgical device 600 having a plurality of tracks 660 with each track 660 including a rail 662 coupled to an outer surface 625 of an elongate shaft or overtube 622 via a flange 664. Generally, the device 600 can be similar to the devices 10, 100, 200, 300, 500 discussed above and have a proximal portion (not shown) with a thinner inner wall than an inner wall 614w of a distal portion 614 of the shaft 622. An inner lumen 624 extends longitudinally through the device 600, in this embodiment axially aligned with a central longitudinal axis A2 of the shaft 622. To help ease insertion of the device 600 into a body of a patient, the device 600 can optionally be advanced through a flexible outer sheath 670 to help prevent the tracks 660 from causing or incurring any damage to the patient and/or to another surgical tool. A person skilled in the art will appreciate that any of the surgical devices described herein can optionally be introduced into a body through a flexible outer sheath.

The outer sheath 670 can be fixedly or removably disposed over the shaft 622, and over the tracks 660 in this embodiment having the tracks 660 radially extending outward from the shaft 622. Generally, the sheath 670 can be configured to form a barrier between an external environment and the shaft 622 to help protect the shaft 622 from fluid and/or other debris that could damage or interfere with proper functioning of the shaft 622 and/or a surgical instrument disposed therein. Non-limiting examples of a sheath can be found in U.S. patent application Ser. No. 12/111,425 titled “Methods and Devices for Maintaining Visibility” filed Apr. 29, 2008, which is hereby incorporated by reference in its entirety.

As will be appreciated by a person skilled in the art, the sheath 670 can have a variety of shapes, sizes and configurations. The sheath 670 can be formed from a variety of materials, e.g., C-Flex® available from Consolidated Polymer Technologies of Clearwater, Fla., and can be formed from a fluid impermeable, biocompatible material. The sheath 670 can be optically clear, translucent, opaque, or any combination thereof. An optically clear sheath can minimize obstruction of the viewing path of a scoping device received in the steering platform 670. If optically clear, the sheath can be formed from non-magnifying 1× material so as to be substantially non-modifying of the view provided by a scoping device disposed therein.

The sheath 670 can have any shape. In this illustrated embodiment, the sheath 670 has an elongate tubular shape having an open proximal end, an open distal end, and an inner pathway extending longitudinally between its proximal and distal ends. The sheath 670 can be disposed around and receive the device 600 within its inner pathway. The sheath's distal and/or proximal ends (not shown) can be respectively secured to the shaft's distal and/or proximal ends (not shown) so as to form a fluid-sealed barrier around the shaft 622. In some embodiments, the sheath 670 can be coupled to the device 600 using an attachment mechanism configured to engage the sheath 670, e.g., at the sheath's proximal end, such as a clip, a clamp, adhesive, a groove, a hook, or any other coupling mechanism appreciated by a person skilled in the art. The attachment mechanism can be located on the shaft 622, the device's handle (not shown), and/or on an introducer device used to introduce the device 600 with the sheath 670 positioned therearound into a body cavity.

The size of the sheath 670 can vary, and the sheath 670 can have a size and shape that can correspond with the size and shape of the shaft 622, with the tracks 670 extending outwardly therefrom, when the sheath 670 is disposed therearound (with or without stretching or flexing of the sheath 670). The sheath 670 can also have any thickness, e.g., 0.015 in. thick. In some embodiments, an internal sheath (not shown) can be disposed inside the inner lumen 624 of the shaft 622. The internal sheath can be flexible and can be made from any material, such as a braided nylon tube, expanded PTFE (polytetrafluoroethylene), or other flexible lubricious, thin-walled material.

As illustrated in FIG. 20, the sheath 670 can optionally include stability threads 671 in a proximal portion 672 thereof. The stability threads 671 can generally be configured to provide stability to the sheath 670 such that when the device 600 is removed from the sheath's inner pathway through the proximal end of the sheath 670, the sheath 670 can remain inserted in the patient's body with the stability threads 671 “bunching” as necessary. A non-limiting embodiment of stability threads include Endopath® Adjustable Stability Threads available from Ethicon Endo-Surgery, Inc. of Cincinnati, Ohio.

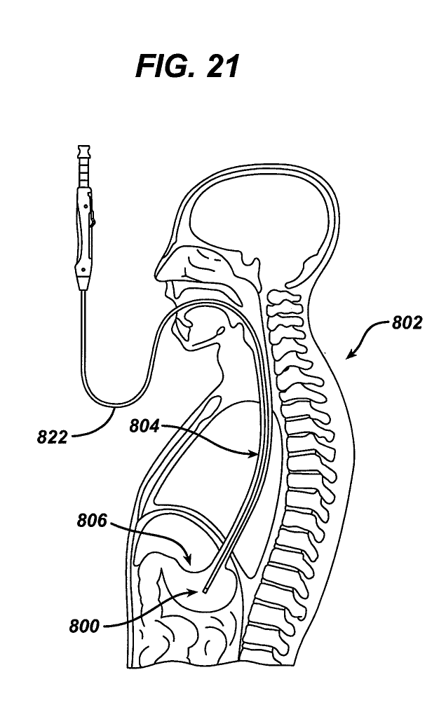

In use, any of the surgical devices having a central column or at least one track as described herein can be introduced into a body of a patient for use in a surgical procedure. The device can be introduced in a variety of ways, such as through a natural orifice, an incision, and/or a trocar, as will be appreciated by a person skilled in the art. FIG. 21 illustrates an exemplary embodiment of a surgical device 800 transorally introduced into a patient 802 with a distal portion of an overtube 822 of the device 800 and at least a portion of a proximal portion of the overtube 822 advanced through an esophagus 804 of the patient 802 into a stomach 806 in an abdominal cavity of the patient 802. Generally, the device 800 can be similar to the devices 10, 100, 200, 300, 500, 600 discussed above with the proximal portion of the overtube 822 having a thinner inner wall than an inner wall of the distal portion of the overtube 822, and with a support structure extending longitudinally through the overtube 822.

The support structure can help transmit a distally directed force applied to the device 800, e.g., from outside the body of the patient 802, to help distally advance the device 800 through the esophagus 804. The distal portion of the overtube 822 can expand the esophagus 804 as necessary to accommodate the diameter of the device 800 at least in a distal portion thereof to help move the distal portion therethrough, thereby also allowing the trailing proximal portion of the overtube 822 to be distally advanced through the esophagus 822. Where the proximal portion is positioned within the esophagus 804, the esophagus 804 can naturally relax, e.g., contract radially inward to its normal, unexpanded state, with the overtube's thinner proximal portion correspondingly contracting radially inward. Any portion of the overtube's proximal portion in a collapsed position can contact an inner wall of the esophagus 804, although some or all of the proximal portion positioned in the esophagus 804 can sufficiently contract to not contact the esophagus 804. When the overtube's distal portion distally exits the esophagus 804 and enters the stomach 806, only the thinner proximal portion of the overtube 822 can be disposed in the esophagus, thereby allowing an entire longitudinal length of the esophagus 804 to relax and experience minimal or no force from the device 800 inserted therethrough. The esophagus 804 and/or the overtube's proximal portion can nevertheless each radially expand outward as necessary to accommodate any one or more surgical tools advanced through the overtube 822. Similarly, the support structure can help transmit a proximally directed force applied to the device 800 to help proximally withdraw the device 800 from the stomach 806 and/or the esophagus 804.

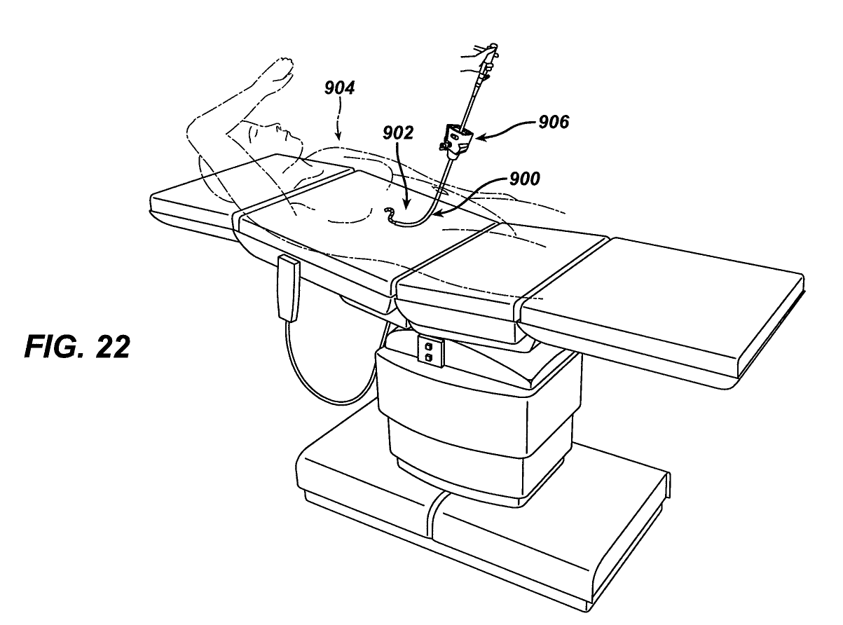

FIG. 22 illustrates another exemplary embodiment with a device 900 having a central column or at least one track being percutaneously introduced into an abdomen 902 of a patient 904 through a trocar 906. Similar to the devices 100, 200, 300, 500, 600, 800 discussed above, the device 900 can have an overtube or elongate shaft having a distal inner wall that is thicker than a proximal inner wall thereof, with the proximal inner wall being configured to radially collapse inward to help the device 900 occupy less space in the patient 904 and reduce forces exerted by the device 900 onto the patient 904.

A person skilled in the art will appreciate that the present invention has application in conventional endoscopic and open surgical instrumentation as well application in robotic-assisted surgery.

The devices disclosed herein can also be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam, and a liquid bath (e.g., cold soak).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A surgical device, comprising:
a flexible elongate shaft having an inner lumen extending therethrough, a proximal portion of the elongate shaft having a wall with a first thickness and a distal portion of the elongate shaft having a wall with a second thickness that is larger than the first thickness; and
a support structure coupled to an interior surface of the elongate shaft and longitudinally extending through the inner lumen of the elongate shaft along a longitudinal length of the elongate shaft.

2. The device of claim 1, wherein the support structure includes a flexible central column extending through the inner lumen in the proximal and distal portions of the elongate shaft.

3. The device of claim 2, further comprising at least one strut radially extending between the central column and the wall in the distal portion of the elongate shaft.

4. The device of claim 3, further comprising a plurality of struts, each of the struts radially extending between the central column and the wall in the distal portion of the elongate shaft, wherein the struts are spaced substantially equidistantly around the central column.

5. The device of claim 2, wherein the central column has at least one cut-out formed therein that extends along a longitudinal length of the central column, the at least one cut-out configured to slidably guide a surgical tool therethrough.

6. The device of claim 2, further comprising a mating member in sliding engagement with the central column; and
an accessory secured to the mating member and configured to receive a surgical instrument therein, wherein the accessory and the mating member are configured to slide as a unit along the central column.

7. The device of claim 1, wherein the support structure includes at least one track extending through the inner lumen in the proximal and distal portions of the elongate shaft, the at least one track coupled to an interior surface of the walls in the distal and proximal portions and configured to engage and guide a surgical instrument disposed through the inner lumen of the elongate shaft.

8. The device of claim 7, further comprising an accessory configured to slide along the track within the inner lumen of the elongate shaft, the accessory having a passageway for passing a surgical instrument.

9. The device of claim 1, wherein the first cross-sectional thickness has a thickness that is in a range of about 10% to 50% of the second cross-sectional thickness.

10. The device of claim 1, wherein the proximal portion of the elongate shaft is radially compressible.

11. A surgical device, comprising:
an overtube having
an insertion section with a proximal portion and a distal portion, the proximal portion having a flexible wall with a first thickness and a first compressive strength, and the distal portion having a flexible wall with a second thickness greater than the first thickness and a second compressive strength greater than the first compressive strength, and
a central column extending through the insertion section, the central column being flexible and being configured to transmit a distally directed force to maneuver the overtube axially.

12. The device of claim 11, wherein the overtube has at least one strut extending radially between the central column and the flexible wall of the distal portion.

13. The device of claim 12, wherein the overtube has a plurality of radial struts extending between the central column and the flexible wall of the distal portion, the plurality of struts defining a plurality of channels in the distal portion equal to the number of struts.

14. The device of claim 12, wherein the central column has at least one cut-out formed in an outer surface thereof, the at least one cut-out configured to slidably guide a surgical tool therethrough.

15. The device of claim 11, further comprising at least one mating member coupled to the central column, the at least one mating member extending through the insertion section of the overtube, and an accessory coupled to the mating member and extending radially relative to the central column.

16. The device of claim 11, wherein the central column has a longitudinal track coupled to an exterior surface thereof, the track being configured to slidably guide a surgical tool therethrough.

17. The device of claim 16, wherein the central column has a passageway extending longitudinally therethrough configured to slidably receive a surgical tool therein.

18. A surgical device, comprising:
an overtube having
an insertion section with a proximal portion and a distal portion, the proximal portion having a flexible wall with a first thickness and a first compressive strength, and the distal portion having a flexible wall with a second thickness greater than the first thickness and a second compressive strength greater than the first compressive strength, and
at least one track coupled to an interior surface of the overtube and extending longitudinally along the insertion section, the at least one track configured to slidably engage a surgical tool disposed through the overtube.

19. The device of claim 18, wherein the overtube has a plurality of tracks coupled to an interior surface thereof, the tracks arranged substantially equidistantly around a circumference of the overtube.

20-23. (canceled)
